(19) United States
(12) Patent Application Publication
(10) Pub. No.: US 2010/0331812 A1
(43) Pub. Date: Dec. 30, 2010

(54) Pharmaceutical Formulations for Iontophoretic Delivery of an Immunomodulator

(75) Inventors: Phillip M. Friden, Bedford, MA (US); Hyun D. Kim, Weston, MA (US); Bireswar Chakraborty, Andover, MA (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: Nitric BioTherapeutics, Inc.

(21) Appl. No.: 12/826,214

(22) Filed: Jun. 29, 2010

Related U.S. Application Data
(60) Provisional application No. 61/269,700, filed on Jun. 29, 2009.

(51) Int. Cl. A61N 1/30; A61K 31/437; A61K 33/04; A61P 35/00; A61P 17/12

(52) U.S. Cl. 604/501; 514/293; 424/711

(57) ABSTRACT

The present invention describes pharmaceutical formulations and methods suitable for iontophoretic delivery of the formulations to a subject. The formulations comprise an immunomodulator, such as imiquimod, and optionally include various agents and excipients. The formulations can be used as a treatment for skin diseases and conditions such as actinic keratosis, basal cell carcinoma and genital warts. The short term iontophoretic delivery of the formulations results in the creation of a depot effect in the skin of the subject, allowing for a sustained delivery. The shortened delivery time minimizes local side effects at the application site.

### CROSS-REFERENCE TO RELATED APPLICATIONS

This application claims the benefit of priority to U.S. Provisional Patent Application No. 61/269,700 filed Jun. 29, 2009, the disclosure of which is incorporated by reference herein as if set forth herein in its entirety.

### BACKGROUND OF THE INVENTION

Imiquimod (mol. mass of 240.30 g/mol; log P: 2.7; pKa: 7.3), is a member of the imidazoquinoline amine family. It is an immunomodulator which displays agonist activity towards toll-like receptors (TLR). Imiquimod is commercially available as a 5% cream (Aldara®, 3M) formulation and is currently approved for treating actinic keratosis, basal cell carcinoma, and genital warts. Typical administration of imiquimod has been through topical, passive application, such as the previously mentioned commercially available cream.

Unfortunately, there are a number of drawbacks to the passive administration of topical creams. For example, topical administration of imiquimod through the use of passive delivery results in limited efficacy. In addition, the requisite high frequency of application (2-5 times per week for up to 16 weeks), dose variability, and unpleasant local side effects at the application site make a topical formulation less than ideal.

Iontophoresis has been known for many years as a means to deliver drugs and cosmetic active agents into the skin for therapeutic purposes. An iontophoretic delivery system is, for example, a drug delivery system that releases drug at a controlled rate to the target tissue upon application. The advantages of systems wherein drug is delivered locally via iontophoresis are the ease of use, relatively safe administration, the ability to finely modulate the dose by changing the time of application and/or the current level and the ability to interrupt administration by simply stopping the current and/or peeling off or removing it from the skin or other body surface whenever an overdosing is suspected. Additionally, due to the relatively short delivery times, formulations with long term exposure issues (such as low or high pH) may be employed. In recent years iontophoretic delivery of drugs has attracted wide attention as a better way of administering drugs for both local and systemic effects. The design of iontophoretic delivery systems can usually be such that the side effects more frequently seen with the systemic administration of conventional dosage forms are minimized.

Iontophoresis involves the application of an electromotive force to drive or repel ions through the dermal layers into a target tissue. Particularly suitable target tissues include those adjacent to the delivery site for localized treatment. Uncharged molecules can also be delivered using iontophoresis via a process called electroosmosis.

Regardless of the charge of the medicament to be administered, an iontophoretic delivery device employs two electrodes (an anode and a cathode) in conjunction with the patient's (or subject's) skin to form a closed circuit between one of the electrodes (referred to herein alternatively as a “working” or “application” or “applicator” electrode) which is positioned at the site of drug delivery and a passive or “grounding” electrode affixed to a second site on the skin to enhance the rate of penetration of the medicament into the skin adjacent to the applicator electrode. Traditionally, iontophoretic drug delivery has been applied to a single, living tissue type, e.g. stratum corneum and dermis. U.S. Pat. No. 6,477,410 issued to Henley et al. describes the use of iontophoresis for drug delivery in the treatment of a variety of diseases. Iontophoresis offers an unexpected enhancement to imiquimod topical formulations by improving permeation, generating a depot effect in the skin (to increase residence time and achieve less frequent application), and minimizing local side effects.

The currently available formulations of imiquimod are formulated for passive topical delivery only and are not intended or suitable for iontophoretic delivery, in which a short-duration of skin exposure allows for the use of non-traditional formulation parameters (such as low or high pH). There is an long felt need in the art to develop an imiquimod formulation which can be delivered quickly and effectively using iontophoresis with better patient acceptance and an improved dosing regimen.

### SUMMARY OF THE INVENTION

The present invention relates to a composition suitable for iontophoresis comprising imiquimod or a pharmaceutically acceptable salt thereof, wherein the concentration of imiquimod is between about 0.1% and 5% by weight, and wherein the pH is between about 3.0 and 5.0.

In one embodiment, the iontophoretic formulation comprises an agent that increases residence time and/or creates a depot effect. In another embodiment, the agent is present at a concentration between about 0.1% and 30% by weight. In another embodiment, the agent is selected from the group consisting of diethylene glycol monoethyl ether, a saturated fatty acid and polyethylene glycol. In another embodiment, the formulation further comprises a buffer system. In another embodiment, the buffer system is selected from the group consisting of a citrate buffer system, a hydrochloride buffer system and an acetate buffer system. In another embodiment the formulation comprises a chelating agent. In another embodiment, the chelating agent is disodium edetate. In another embodiment, the formulation comprises an antioxidant. In another embodiment, the antioxidant is selected from the group consisting of BHA, BHT, sodium sulfite and an amino acid. In another embodiment, the formulation comprises an emollient. In another embodiment, the emollient is glycerine. In another embodiment, the formulation comprises a surfactant. In another embodiment, the surfactant is polysorbate 80.

In on embodiment the present invention relates to a method of treating a skin disease or condition of a patient or subject, wherein a composition suitable for iontophoresis comprising imiquimod or a pharmaceutically acceptable salt thereof is applied. In another embodiment, the concentration of imiquimod in the formulation is between about 0.1% and 5% by weight, and the pH is between about 3.0 and 5.0. In another embodiment, the skin disease or condition is selected from actinic keratosis, basal cell carcinoma, and genital warts. In another embodiment, the present invention relates to a method wherein the formulation further comprises at least one or more of a chelating agent, an emollient, a surfactant and a buffer system.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 depicts the chemical structure of Imiquimod.

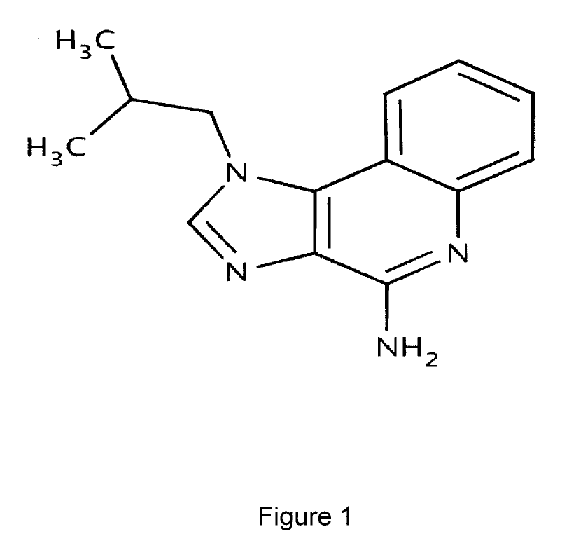

FIG. 2, comprising FIGS. 2A and 2B, depicts the in vitro permeation profile of Imiquimod. FIG. 2A depicts the increase in the cumulative amount of drug at 24 hours from 2.24 μg/cm² for passive to 22.41 μg/cm² upon the application of iontophoresis. FIG. 2B depicts the quantification of total drug levels in the skin.

FIG. 3 depicts the amount of imiquimod in the stratum corneum (SC), underlying skin, and whole skin (stratum corneum plus underlying tissue) over time using the 0.3% w/w formulation.

FIG. 4 depicts the amount of imiquimod in whole skin (stratum corneum plus underlying tissue) over time using the 2% w/w formulation.

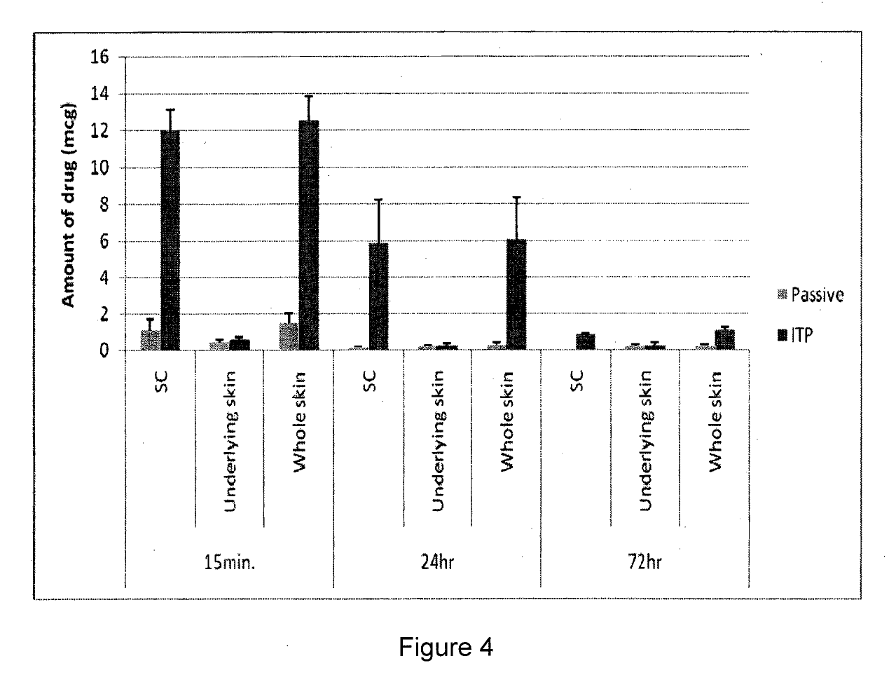

### DETAILED DESCRIPTION

The present invention provides compositions suitable for iontophoretic delivery, comprising an immunomodulator, preferably imiquimod, that is useful for the treatment of skin disease. The present invention is based on the discovery that treatment of skin disease with the present invention using iontophoresis results in the formation of a reservoir of the drug in the stratum corneum layer of the skin. This reservoir disperses into the lower layers of the dermis and epidermis over time, where the targeted Langerhans cells are located, resulting in sustained release of the drug to its effective locations. By providing short application periods of immunomodulator via iontophoresis, the present invention achieves a significantly greater effect as compared to a much longer, passive surface application of the currently available topical creams, without the negative side effects.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein, a “nail” includes reference to the whole nail or any portion of the nail, including the nail plate, the nail bed, the cuticle, the nail folds, the lunula, the matrix, and the hyponychium.

As used herein, a “permeation enhancer” or “penetration enhancer” is a material which achieves permeation enhancement or an increase in the permeability of the skin and/or nail to a pharmacologically active agent.

As used herein, the term “pharmaceutically acceptable carrier or excipient” means any non-toxic diluent or other formulation auxiliary that is suitable for use in iontophoresis.

As used herein, a “therapeutically effective amount” is an amount of drug, such as an immunomodulator, that is sufficient to prevent development of or alleviate to some extent one or more of a patient's symptoms of the disease being treated.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a skin disease in a patient.

### Description

The present invention provides stable formulations of imiquimod suitable for iontophoretic delivery to the treatment site of a subject in need thereof. Imiquimod can be used to treat a variety of diseases, including but not limited to actinic keratosis, warts, and basal cell carcinoma. The present invention is based on the discovery that iontophoresis has the potential to significantly enhance the penetration of imiquimod into the epidermis and dermis. The level of exposure of Langerhans cells, located in the epidermis and dermis, to imiquimod may determine clinical efficacy of formulation.

As contemplated and demonstrated herein, clinical data illustrate that levels of drug retention in the stratum corneum differs using the present invention from current topical techniques. The results herein demonstrate the significant retention of the drug in the stratum corneum, resulting in a reservoir effect and stable long term release. The results herein further demonstrate the effectiveness of a single iontophoretic application over current topical applications.

### Formulations

Several formulation criteria are addressed to achieve optimal iontophoretic delivery of imiquimod. These include minimizing competing charges in the formulation and maintaining a viscosity that is as low as possible to allow retention in an applicator without unduly affecting the flow of the charged drug molecules along the electric field. The present formulations maintain the drug in an ionized state at a high concentration and are also non-irritating for short duration exposure. The formulations remain stable under conventional storage conditions as well as during iontophoresis. An exemplary formulation is one that is a single phase (such as a water soluble gel) that contains a high concentration of the drug in an ionized state.

The present invention includes stable formulations for iontophoretic delivery of imiquimod for the treatment of diseases or conditions such as actinic keratosis, warts, and basal cell carcinoma. In an exemplary embodiment of the present invention, an iontophoretic device is used to facilitate delivery of the formulation into and through the skin.

As contemplated herein, the present invention includes a formulation suitable for iontophoretic delivery comprising an immunomodulator. Preferably, the immunomodulator is imiquimod or a pharmaceutically acceptable salt thereof. Different salt forms may be used based on the suitability of the drug for iontophoresis. In one embodiment, the amount of the imiquimod or pharmaceutically acceptable salt is between about 0.1% and 5% by weight. The pH and conductivity may be considered so that the salt can be ionized at the selected pH and have sufficient conductivity for iontophoresis. In another embodiment, the formulation comprises an effective amount of imiquimod and has a pH between about 3 and 5, allowing for optimal solubility and maximal charge of the imiquimod molecule.

In another embodiment, the formulation may include one or more suitable thickeners. The thickener may be nonionic and have minimal influence on the migration of the immunomodulator under electric current based on viscosity, material electric charge or porosity. Exemplary thickeners may include, without limitation, cellulose derivatives, pluronics, polyvinyl acetate, polyvinyl pyroolidones and combinations thereof.

In one embodiment, the formulation may comprise one or more suitable agents capable of increasing residence time and building a depot effect for at least 24 hours, allowing the drug to be released from the epidermis slowly. Exemplary agents may include, without limitation, glycol ethers, polyethylene glycols, propylene glycol, poloxamer, saturated fatty acids and combinations thereof. In a still further embodiment, the agent may be present in an amount from about 0.1% to 30% w/w.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition.

In one embodiment, the formulation may comprise a suitable surfactant and/or solubilizer. Exemplary surfactants may include, without limitation, non-ionic surfactants, glycol ethers, polyethylene glycols, propylene glycol, poloxamer, saturated fatty acids and combinations thereof. In a further embodiment, the surfactant or solubilizer may be polysorbate 80 (Tween 80) and/or propylene glycol. In a further embodiment the polysorbate 80 and/or propylene glycol may be present in an amount from about 0.1% to 30% w/w.

In one embodiment, the formulation may comprise an emollient. Preferably the emollient may be glycerin. In a further embodiment the emollient may be present in an amount from about 1% to 30% w/w.

In one embodiment, the formulation may comprise an agent that is a suitable stabilizer, a chelator and/or antioxidant. Preferably the antioxidant butylated hydroxyl anisole may be used. Exemplary chelators include, without limitation, EDTA, BHA, BHT, sodium sulfite, amino acids and combinations thereof. Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

In yet another embodiment, the formulation may comprise a suitable preservative. Preferably the preservative may be benzalkonium chloride. In a further embodiment the benzalkonium chloride may be present in the amount of 0.01% to 0.02% w/w in the formulation. Other exemplary preservatives include, without limitation, methyl paraben/propyl paraben, phenyl ethyl alcohol, benzoic acid, benzalkonium chloride and combinations thereof.

In another embodiment, the formulation may comprise a mosturizing agent. Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol. Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quatemium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid; sandalwood (santalum album) oil, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

Non-limiting examples of additional compounds and agents that can be used with the compositions of the present invention include vitamins (e.g. D, E, A, K, and C), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), dyes and color ingredients (e.g. D&C blue no. 4, D&C green no. 5, D&C orange no. 4, D&C red no. 17, D&C red no. 33, D&C violet no. 2, D&C yellow no. 10, D&C yellow no. 11 and DEA-cetyl phosphate), antimicrobial agents (e.g., triclosan and ethanol), and fragrances (natural and artificial).

In yet another embodiment, the formulation may comprise a suitable buffer system to control pH. Preferably the buffer system may consist of citrate, hydrochloride or acetate. In a further embodiment the buffer system may maintain pH between about 3.0 and 5.0.

According to an aspect of the present invention, the formulation can be administered iontophoretically. In one embodiment, the current density may be between about 0.01 and 1.0 mA/cm². In another embodiment the formulation can be administered for a time ranging from 5 minutes to 8 hours. In a further embodiment the amount of current and time with which the formulation is iontophoretically administered will depend on the therapeutic condition and surface area being treated.

Iontophoretic compositions according to the current invention may be provided in a variety of formulations. For example, as liquids, creams, salves, ointments, gels, or eye drops. In certain embodiments iontophoretic compositions may be contained in a reservoir. In other embodiments such reservoirs may comprise patches, for example hydrogel patches. However, it will be understood by one of skill in the art that an important characteristic of an iontophoretic composition, and of a reservoir comprising said iontophoretic composition, is to be capable of conducting electricity. Some examples of polymers that may comprise a reservoir or patch include but are not limited to aqueous swollen cross linked water soluble polymers such as polyethylene oxide, polyvinyl pyrrolidone, polyvinyl alcohol, polyacrylamide or polyethylene glycol. Additional composition for reservoirs are described in detail in U.S. Pat. Nos. 6,862,473, 6,858,018, 6,629,968, 6,377,847, 5,882,677, and 5,738,647, and examples of patch design are described in U.S. Patent Applications 20030175328 or 20030175333 or PCT publication WO 2004062600, all incorporated herein by reference in their entirety.

It should be understood that the formulations and compositions that would be useful in the present invention are not limited to the particular formulations and compositions that are described herein.

### Devices for Iontophoretic Delivery

In certain embodiments, iontophoresis devices for use according to the invention may comprise an apparatus that is carried on the body during treatment. For example, the apparatus man be worn in clothing or adhered to a portion of the body. Thus, in certain aspects the apparatus may comprise a power source that is placed at distance from the treatment site, but connected via to the site via wires or an interconnect. The LidoSite iontophoresis device available from Vyteris, Inc (www.vyteris.com) is one such example. In the case where the immunomodulator compositions are applied to the eyes, the wires for the apparatus may be supported, for example by eyeglasses.

Some examples of iontophoresis devices that may be used according to the current invention include, but are not limited to, the Phoresor II Auto, the Phores PM900, the Empi Dupel, the apparatuses described in U.S. patent applications 20050113738, 20050070840, 20040167460, 20040116964, 20040064084, 20040039328, 20030135150 and U.S. Pat. Nos. 6,731,987 and 6,064,908, the entire disclosures of which are incorporated by reference herein as if set forth herein in their entirety.

In certain embodiments, it is contemplated that an immunomodulator composition may be applied separately from the electrode of the iontophoresis apparatus. For example, in certain embodiments the immunomodulator composition may be topically applied followed by application of the iontophoresis electrode. In certain cases the immunomodulator compositions may be applied multiple times to the electrode area during iontophoresis to enhance the efficacy of the treatment.

### Administration and Dosage

As contemplated herein, the present invention includes formulations suitable for iontophoretic delivery comprising an immunomodulator for the treatment of skin diseases of a patient or subject in need thereof. In certain embodiments, the patient or subject is a mammal, and preferably a human. A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. Dosage regimens may be adjusted to provide the optimum therapeutic response. For example, several divided doses may be administered daily or the dose may be proportionally reduced as indicated by the exigencies of the therapeutic situation. The regimen of administration may affect what constitutes an effective amount of the immunomodulator. Further, several divided dosages, as well as staggered dosages may be administered daily or sequentially. Further, the dosages of the therapeutic formulations may be proportionally increased or decreased as indicated by the exigencies of the therapeutic or prophylactic situation. Administration of the compositions of the present invention to a patient may be carried out using known procedures, at dosages and for periods of time effective to treat skin diseases in the patient.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient. In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors understood in the medical arts. A non-limiting example of an effective dose range for a therapeutic compound of the invention is from about 0.1% to 5.0% by weight of the formulation. One of ordinary skill in the art will understand the relevant factors in determining the effective amount of the therapeutic compound without undue experimentation.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of skin diseases in a patient.

In one embodiment, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. Thus, the invention should not be construed to be limited to any particular dosage regime and the precise dosage and composition to be administered to any patient will be determined by the attending physical taking all other factors about the patient into account.

In one embodiment, the present invention is directed to a packaged pharmaceutical composition comprising a container holding a therapeutically effective amount of a compound of the invention, alone or in combination with a second pharmaceutical agent, and instructions for using the compound to treat, prevent, or reduce one or more symptoms of skin disease in a patient.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Those skilled in the art will recognize, or be able to ascertain using no more than routine experimentation, numerous equivalents to the specific procedures, embodiments, claims, and examples described herein. Such equivalents are considered to be within the scope of the invention. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present invention.

### EXAMPLES

The invention is now described with reference to the following Examples. These Examples are provided for the purpose of illustration only, and the invention is not limited to these Examples, but rather encompasses all variations which are evident as a result of the teachings provided herein.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

As demonstrated herein, the amount of drug in the stratum corneum increased under iontophoretic conditions compared to passive diffusion. Though the levels of imiquimod in the stratum corneum increased with the application of iontophoresis, a similar trend was not observed for the levels of drug in the underlying skin. For both passive and iontophoretic conditions, similar levels of the drug permeated past the stratum corneum and into the underlying skin tissue. For both passive and iontophoretic conditions, the majority of imiquimod accumulated in the stratum corneum, but the levels achieved are significantly enhanced by iontophoresis. Additionally, the application of iontophoresis formed a depot in the stratum corneum with a residence time of more than 72 hr thereby increasing the amount of drug that could be delivered into the skin. This effect allows a large amount of the drug to be quickly administered to the stratum corneum, from which it gradually diffuses into the underlying skin. The result is a larger, faster dosage that allows more effective delivery of the drug to the underlying layers combined with a decrease in necessary exposure time to the irritable surface layer. Irritation or redness of the skin was not observed in any of the in vivo studies. The level of drug accumulation in the underlying skin is not impacted by iontophoresis, and remains at a clinically effective and non-toxic level, similar to passive diffusion. Additionally, the amount of drug that permeates through the skin is significantly increased by iontophoresis (approximately 10-fold).

Imiquimod was demonstrated to induce in vitro production of various cytokines and related products. Imiquimod goes through a quick metabolism process (via hydroxylation). Two imiquimod metabolites are also active and induce cytokine production. Effective concentrations of imiquimod necessary to induce IFN-αproduction in cell cultures (specifically, dendritic cells) are between 0.1 and 5.0 μg/mL and can be taken to indicate the potential for similar in vivo activity (Schon and Schon, Brit. J. Derm. 157:8-13, 2007). Alternatively, imiquimod dose ranges of 25 to 50 ug/mL were observed to induce apoptosis of tumor cells in vitro. Based on these observations, the 15 min iontophoresis at 0.2 mA/cm² delivered approximately 14 ug imiquimod to the skin, which is within the range of effective dosage.

### Example 1

### Solubility Study of Solvent Combinations

Without wishing to be bound by any particular theory, it is believed that the development of a suitable formulation for delivery of imiquimod into the skin by iontophoresis would require the drug to permeate through the stratum corneum and into the underlying epidermis/dermis, where it should remain for a prolonged period (hours to days).

Achieving a long residence time may be possible using a saturated fatty acid, diethylene glycol monoethyl ether (Transcutol P; Gattefosse Co.), propylene glycol, PEG 400, or combinations thereof. A solubility study of combinations of solvents to achieve maximum solubility of imiquimod in solution was designed and is depicted in Table 1.

TABLE 1
Composition and Solubility of Imiquimod Formulations (w/w)

| Sample | Propylene | | | | Isopropyl | | pH adjustment | Solubility |
|---|---|---|---|---|---|---|---|---|
| # | glycol | PEG 400 | Transcutol P | Tween 80 | myristate | Glycerin | to ~3.6 | (mg/ml) |
| 1 | — | 20 | — | 3 | — | 10 | 1N HCl acid | 0.417 |
| 2 | 10 | 20 | — | 3 | — | 10 | 1N HCl acid | 0.348 |
| 3 | 10 | 20 | 20 | 3 | — | 10 | 1N HCl acid | 0.857 |
| 4 | 10 | 20 | — | 3 | — | 10 | 1N Acetic acid | 3.003 |
| 5 | 10 | 20 | 20 | 3 | — | 10 | 1N Acetic acid | 26.234 |

It was observed that a combination of 20% PEG 400, 10% propylene glycol, 20% Transcutol® P, 3% Tween 80 and 10% glycerin (w/w, final pH 3.6 adjusted with acetic acid) results in a maximum solubility of approximately 26 mg/ml.

### Example 2

### Epidermal, Dermal, and Transdermal Delivery of a 0.3% w/w Imiquimod Formulation in Hairless Rat Skin In Vitro

Transdermal drug delivery offers an appealing alternative to invasive hypodermic needles and the safety and bioavailability disadvantages often associated with oral drug delivery. However, the stratum corneum, the outermost layer of skin, is a formidable barrier to many compounds. As a result, effective permeation through skin is generally limited to small, lipophilic molecules. To enhance the permeation of macromolecules, as well as hydrophilic and hydrophobic small molecules, techniques such as iontophoresis are shown herein to enhance delivery.

The effect of iontophoresis on the permeation profile of imiquimod across hairless rat skin in vitro was examined. The solubility of imiquimod was tested in various solvent matrices and a formulation with acceptable solubility was employed for further in vitro and in vivo studies as described herein. The following methods were performed.

For the in vitro studies, Franz diffusion cells were employed. Hairless rats were euthanized by carbon dioxide asphyxiation and the abdominal skin area was excised and carefully cleaned to remove excess fat. The skin was then mounted onto Franz diffusion cells. Citrate buffer with 20% ethanol was employed as the receptor buffer and the drug formulation was placed in the donor chamber. For iontophoretic delivery, a silver/silver chloride electrode system was used. A current density of 0.2 mA/cm² was applied for 15 min (anodal iontophoresis) after which passive diffusion was allowed to occur for 24 hr in the presence of Imiquimod formulation. Samples were taken at predetermined time points and the receptor chamber was replenished with fresh receptor buffer accordingly. At completion, the skin samples were dismounted and washed with the receptor buffer to remove excess drug. Tape stripping and skin extraction studies were performed to quantify the amount of the drug in the skin. All the samples were analyzed via HPLC (C18 column; mobile phase: acetonitrile/50 mM ammonium acetate (70:30); injection volume: 10 μl UV detection at 250 nm).

Using the above methods, various excipients were tested to prepare a formulation with sufficient solubility for imiquimod. A solubility level of about 0.3% was achieved, as indicated by HPLC results, with a formulation comprising about 3 mg/mL imiquimod in about 30% w/w PEG 400, 10% w/w glycerin, and 3% w/w polysorbate 80 at pH 4.

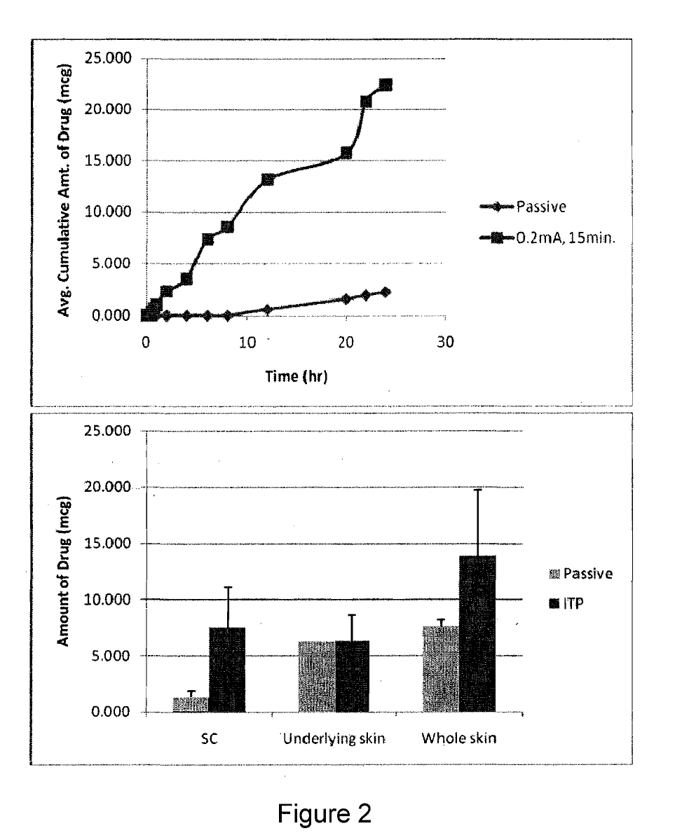

From the in vitro studies, it was observed that imiquimod has a passive permeation of approximately 2 μg/cm² at the end of 24 hr. Following the application of iontophoresis for 15 min, the lag time was decreased and the permeation increased to approximately 22 pg/cm (FIG. 2A). At the end of the in vitro study, the skin samples were subjected to tape stripping and skin extraction to quantify the drug levels in the stratum corneum and the underlying skin, respectively. The amount of drug in the stratum corneum was 1.31 μg and 7.53 μg for passive and iontophoretic conditions, respectively (FIG. 2B). Though the levels of imiquimod in the stratum corneum increased with the application of iontophoresis, a similar trend was not observed for the levels of drug in the underlying skin. For passive conditions, 6.27 μg of the drug permeated past the stratum corneum and into the underlying skin tissue. Similar levels (6.31 μg) were observed for iontophoretic conditions as well.

The results presented herein demonstrate that for both passive and iontophoretic conditions, the majority of imiquimod accumulates in the stratum corneum and that the levels achieved are significantly enhanced by iontophoresis. Much less drug accumulates in the underlying skin, and those levels are not impacted by iontophoresis. Additionally, the amount of drug that permeates through the skin is significantly increased by iontophoresis (approximately 10-fold).

### Example 3

### Quantification of an Imiquimod Skin Depot in Hairless Rats In Vivo Using a 0.3% w/w Imiquimod Formulation

The effect of iontophoresis on its ability to enhance delivery of 0.3% w/w imiquimod (pH 4.0) formulation in vivo into the stratum corneum and the underlying skin (lower epidermis and dermis) was examined. The following methods were performed.

Hairless rats were anesthetized using Ketamine and Xylazine. An area on the abdomen was marked and cleaned. Drug formulation was loaded onto an iontophoretic drug cartridge and was secured on the area with tape. For iontophoretic delivery, the cartridge was connected to a current source and a TransQ iontophoretic patch (Iomed) served as the counter electrode. A current density of 0.2 mA/cm² was applied for 15 min. At the end of 15 min, the drug loaded cartridges were removed and excess drug on the site was removed. Tape stripping (3M Transpore™ tape) was performed to quantify the drug levels in the stratum corneum. Transepidermal water loss (TEWL) measurements were also taken to ensure the complete removal of the stratum corneum. The rats were then euthanized and the skin samples were excised for further analysis. A skin extraction assay was performed to quantify the drug levels in the underlying skin immediately after 15 min iontophoresis or passive. The same procedure was repeated at the end of 24 hr and 72 hr after the application of the drug to determine the clearance of drug from the skin.

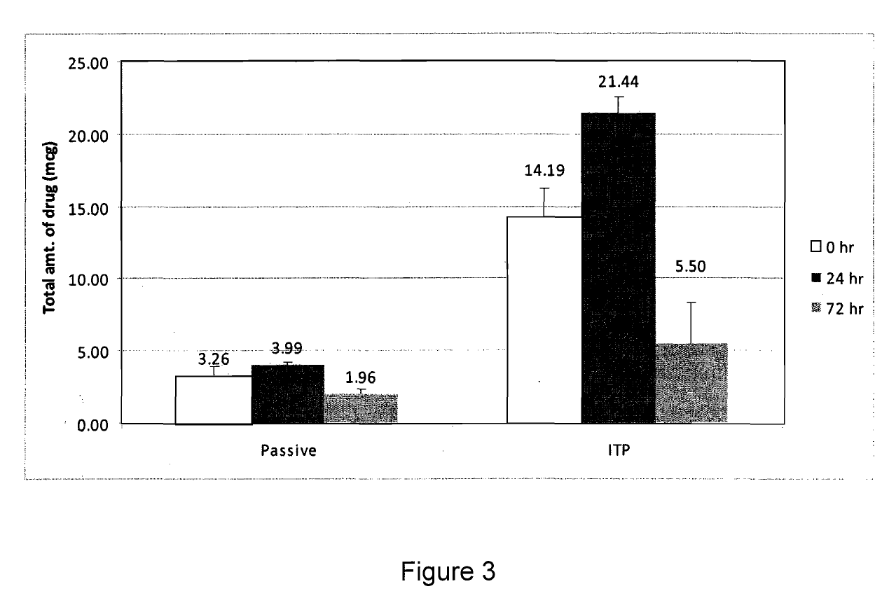

Having achieved a 10-fold increase in permeation of the drug with the application of iontophoresis in vitro, the delivery profile was characterized in vivo. After about 15 min of treatment, passive skin samples had 1.07 μg of the drug in the stratum corneum and 0.40 μg in the underlying skin. Iontophoresis subjected skin samples had 10-fold higher drug levels in the stratum corneum (11.91 μg), but the levels in the underlying tissue did not follow the same trend (0.591 μg). A similar pattern was observed for drug levels at 24 hr and 72 hr from the time of treatment. Drug levels in the stratum corneum and skin decreased with time, but there did not appear to be an accumulation of the drug in the underlying skin once it diffused out from the stratum corneum. At 72 hr, drug was still detected in both the stratum corneum and underlying skin for the iontophoresis skin samples, but no drug was detected in the stratum corneum of the passive skin samples (FIG. 3). A small amount of drug was also present in the underlying tissue for the passive delivery samples at this time point.

The results presented herein demonstrate that for both passive and iontophoretic conditions, the majority of the imiquimod accumulates in the stratum corneum, but that the levels achieved are enhanced by iontophoresis. Far less drug accumulates in the underlying skin, and those levels are equivalent for both passive delivery and iontophoresis. The in vitro data suggests that iontophoresis drives more drug through the skin, but in vivo that additional drug appears to be rapidly cleared from the tissue, leaving a basal level that is similar to that achieved by passive delivery. Thus, the application of iontophoresis increased the amount of drug that could be delivered into the skin to form a depot in the stratum corneum with a residence time of more than 72 hr while at the same time allowing for a long term sustained dose to the underlayer, as needed for treatment, with only short term surface exposure. Irritation or redness of the skin were not observed in any of the in vivo Examples.

### Example 4

### Formation and Desorption of an Imiquimod Skin Depot In Vivo Using a 2% w/w Imiquimod Formulation containing Transcutol® P

In this example the formation and pharmacokinetics of an imiquimod skin depot following iontophoretic delivery in an in vivo hairless rat model were examined.

A topical delivery system containing 2% w/w drug formulation (consisting of 20% PEG 400, 10% propylene glycol, 20% Transcutol P, 3% Tween 80 and 10% glycerin; in w/w, final pH 3.6) was employed. The formulation was loaded into a cartridge and iontophoresis (0.2 mA/cm², 15 min) was used to deliver the drug into the skin using the hairless rat model as described herein. A passive delivery control was run for comparison. Immediately after treatment, tape stripping and skin extraction were performed to quantify the drug levels in the stratum corneum and the underlying skin, respectively. Transepidermal water loss (TEWL) measurements were taken to ensure the removal of the stratum corneum. The same procedure was repeated 24 and 72 hrs after drug application to determine the clearance of drug from the skin.

The results from this Example demonstrate that a depot formed in the skin with both iontophoretic and passive delivery. However, the levels of imiquimod in both the stratum corneum and underlying skin were significantly higher in the iontophoresis group. Total skin drug levels of 14.19±2.07 μg and 3.26±0.63 μg were observed after 15 min of iontophoretic and passive delivery, respectively. Most of the drug was concentrated in the stratum corneum layer, with lower drug levels in the underlying skin. Drug levels in both skin compartments decreased gradually as a function of time. By 72 hrs, the drug levels decreased from an initial amount of 14.19±2.07 μg to 5.50±2.76 μg for iontophoretic delivery and 3.26 μg±0.63 μg to 1.96±0.37 μg for passive delivery, respectively. No drug was detected in the underlying skin for both conditions at 72 hrs.

The results presented herein show that iontophoretic delivery of imiquimod resulted in a depot in the stratum corneum. By 72 hrs, the drug was still detected in the stratum corneum layer but was completely desorbed from the underlying skin. Similar kinetics, but at much lower levels, were observed with passive delivery.

### Example 5

### Effective Concentration of Imiquimod Delivered into Skin

Imiquimod has been shown in animals and human cell lines to induce cytokine production from monocytes and macrophages (including IFN-α, IL-6, TNF-α). The topical cream of imiquimod was designed to produce local cytokine induction by targeting Langerhan cells within the skin. In addition, by preventing/reducing systemic absorption, systemic side effects can be minimized. However, the major challenge for the imiquimod cream is the transport of the drug into and through the skin.

Imiquimod has been demonstrated to induce in vitro production of various cytokines and related products. Imiquimod goes through a quick metabolism process (via hydroxylation). Two imiquimod metabolites are also active and induce cytokine production. Effective concentrations of imiquimod necessary to induce IFN-α production in cell cultures (specifically, dendritic cells) are between 0.1 and 5.0 μg/mL and can be taken to indicate the potential for similar in vivo activity (Schon and Schon, Brit. J. Derm. 157:8-13, 2007). Alternatively, imiquimod dose ranges of 25 to 50 ug/mL were observed to induce apoptosis of tumor cells in vitro. Based on these observations, 15 min iontophoresis at 0.2 mA/cm² delivered approximately 14 ug imiquimod to the skin, which is within the range of effective dosage.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

The disclosures of each and every patent, patent application, and publication cited herein are hereby incorporated herein by reference in their entirety.

What is claimed:

1. A composition suitable for iontophoretic delivery to a treatment site of a patient in need thereof, comprising imiquimod or a pharmaceutically acceptable salt thereof at a concentration between about 0.1% and 5% by weight, wherein the pH of the composition is between about 3 and 5.

2. The composition of claim 1, further comprising an agent at a concentration of between about 0.1% and 30% by weight, wherein the agent increases residence time via a depot effect within the skin of the patient.

3. The composition of claim 2, wherein the agent is selected from the group consisting of diethylene glycol monoethyl ether, a saturated fatty acid and polyethylene glycol.

4. The composition of claim 3, wherein the depot effect within the skin is localized in the stratum corneum.

5. The composition of claim 1, further comprising a buffer system, wherein the buffer system is selected from the group consisting of a citrate buffer system, a hydrochloride buffer system and an acetate buffer system.

6. The composition of claim 1, further comprising a chelating agent.

7. The composition of claim 6, wherein the chelating agent is disodium edetate.

8. The composition of claim 1, further comprising an antioxidant.

9. The composition of claim 8, wherein the antioxidant is selected from the group consisting of BHA, BHT, sodium sulfite and an amino acid.

10. The composition of claim 1, further comprising an emollient.

11. The composition of claim 10, wherein the emollient is glycerin.

12. The composition of claim 1, further comprising a surfactant.

13. The composition of claim 12, wherein the surfactant is polysorbate 80.

14. The composition of claim 1, wherein the composition is a single phase.

15. The composition of claim 14, wherein the composition is a water soluble gel.

16. A method of treating a skin disease or condition of a patient in need thereof, comprising iontophoretically administering to the patient a composition comprising imiquimod or a pharmaceutically acceptable salt thereof at a concentration between about 0.1% and 5% by weight, wherein the pH of the composition is between about 3 and 5.

17. The method of claim 16, further comprising increasing residence time of the composition within the skin of the patient.

18. The method of claim 17, wherein the depot effect within the skin is localized in the stratum corneum.

19. The method of claim 18, wherein the depot effect localized in the stratum corneum has a residence time of more than about 72 hours.

20. The method of claim 16, wherein the skin disease or condition is actinic keratosis, basal cell carcinoma or genital warts.

21. The method of claim 16, wherein the composition further comprises at least one or more of a chelating agent, an antioxidant, an emollient, a surfactant and a buffer system.
